(19) United States
(12) Patent Application Publication
(10) Pub. No.: US 2022/0341932 A1
(43) Pub. Date: Oct. 27, 2022

(54) ANTIBODY ASSAY FOR SARS-CoV-2

(71) Applicants: Arizona Board of Regents on behalf of Arizona State University, Scottsdale, AZ (US); Halberd Corporation, Jackson Center, PA (US)

(72) Inventors: Qiang Chen, Chandler, AZ (US); Haiyan Sun, Chandler, AZ (US); Collin Bradley Jugler, Mesa, AZ (US); Adrian Esqueda, Tempe, AZ (US); Mitchell Steven Felder, Hermitage, PA (US)

(21) Appl. No.: 17/726,109

(22) Filed: Apr. 21, 2022

Related U.S. Application Data
(60) Provisional application No. 63/178,354, filed on Apr. 22, 2021.

(51) Int. Cl. G01N 33/569; G01N 33/577; G01N 33/533

(52) U.S. Cl. CPC G01N 33/56983; G01N 33/577; G01N 33/533; G01N 2446/00; G01N 2333/165

(57) ABSTRACT

An embodiment provides a method for detection of viral antigen for the COVID-19 virus, including: obtaining a body fluid from a patient; introducing the body fluid to at least one binding antibody, wherein the at least one binding antibody binds to an antigen of the SARS-CoV-2 spike (S) protein and comprises an indicator; forming a viral antigen—antibody complex; and determining the presence of the viral antigen—antibody complex. Other aspects are described and claimed.

### CROSS REFERENCE TO RELATED APPLICATION

This application claims priority to U.S. Provisional Patent Application Ser. No. 63/178,354, filed on Apr. 22, 2021, and entitled “ANTIBODY ASSAY FOR SARS-CoV-2,” the contents of which are incorporated by reference herein.

### FIELD

This application relates generally to a diagnostic test for immunity for SARS-CoV-2, and, more particularly, to an assay of antibodies of a patient for SARS-CoV-2.

### BACKGROUND

The instant application contains a Sequence Listing which has been submitted electronically in ASCII format and is hereby incorporated by reference in its entirety. Said ASCII copy, created on Apr. 22, 2021, is named 995-015P-SL.txt and is 7 KB in size.

Coronaviruses represent a group of viruses that may lead to respiratory tract infections. These infections may range from mild to lethal. Coronaviruses may cause severe acute respiratory syndrome (SARS) and Middle East respiratory syndrome (MERS). A novel coronavirus (COVID-19) has led to a global pandemic causing a public health and economic crisis. Severe acute respiratory syndrome coronavirus 2 (SARS-CoV-2) is the strain of coronavirus that causes coronavirus disease 2019. Transmission may be through close contact of individuals and via respiratory droplets such as coughs or sneezes. Faster and more accurate methods of identifying and treating individuals infected with COVID-19 could mitigate the global pandemic.

### BRIEF SUMMARY

In summary, one embodiment provides a method for detection of viral antigen for the COVID-19 virus, comprising: obtaining a body fluid from a patient; introducing the body fluid to at least one binding antibody, wherein the at least one binding antibody binds to an antigen of the SARS-CoV-2 spike (S) protein and comprises an indicator; forming a viral antigen—antibody complex; and determining the presence of the viral antigen—antibody complex.

Another embodiment provides a method for detection of immunity for the COVID-19 virus, comprising: obtaining a body fluid from a patient, wherein the body fluid comprises a viral antigen—primary antibody complex of SARS-CoV-2 spike protein; introducing the body fluid to at least one secondary antibody, wherein the at least one secondary antibody binds to the primary antibody of the SARS-CoV-2 spike (S) protein and an indicator; forming an antigen—primary antibody—secondary complex; and determining the presence of the antigen—primary antibody—secondary complex, wherein the indicator correlates to a level of immunity of the patient.

A further embodiment provides a method for detection of immunity for the COVID-19 virus, comprising: obtaining a body fluid from a patient; introducing the body fluid to a lateral flow device comprising at least one binding antibody, wherein the at least one binding antibody binds to an antigen of the SARS-CoV-2 spike (S) protein and comprises a fluorescent tag; forming an antigen-antibody complex; and determining the presence of the antigen-antibody complex, wherein a fluorescent signal correlates to a level of immunity of the patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example method for an assay of patient antibodies for SARS-CoV-2 using a monoclonal antibody.

FIG. 2 illustrates an example fluorescence plot the base human IgG antibody against spike proteins not specific to Covid-19 and of a monoclonal antibody specific for the spike protein of SARS-CoV-2.

FIG. 3 illustrates an example fluorescence plot of a fluorescent antibody and the normal human spike protein antibody (non-specific) plotted against the actual antibody concentration (as opposed to RBD concentration as in FIG. 2).

FIG. 4 illustrates an example antibody binding methodology for the detection of Covid-19 immunity.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a rapid and accurate assay of COVID-19 antibodies in a patient. For example, a patient may be tested to see if a vaccine or titer is necessary.

Accordingly, an embodiment provides a method for determining the presence of COVID-19 immunity in body fluid of a patient. The identification may be rapid in time. The identification may be from a patient's body fluid. The body fluid may be blood, CSF (cerebrospinal fluid), mucus, saliva, or any bodily fluid. The body fluid may be from a patient which may contain COVID-19 antibodies. In an embodiment, the body fluid may be exposed to at least one binding antibody. In an embodiment, a treatment may be applied to the body fluid. In an embodiment, a treatment may comprise exposing the body fluid to a binding antibody. The binding is to an antigen specific to the spike protein of SARS-CoV-2 or another region of COVID-19. In an embodiment, the method may determine the presence or absence of the antigen-antibody complex. The binding antibody may include, for example, a fluorescent antibody, a luminous antibody, combinations thereof, or the like. The method may utilize different techniques for determining the presence or absence of the antigen-antibody complex as described below. In other words, an antibody may be used as a viral load gradient diagnostic assay which may allow for a healthcare provider to determine if a patient has sufficient antibodies in a decision for a treatment plan.

The illustrated example embodiments will be best understood by reference to the figures. The following description is intended only by way of example, and simply illustrates certain example embodiments.

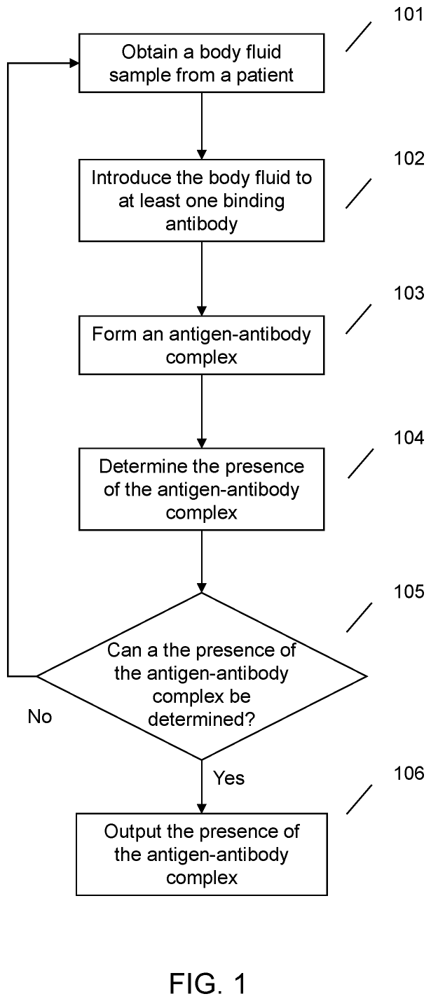

Referring to FIG. 1, an example device and method for determining the presence of COVID-19 antibodies in a body fluid. The body fluid may contain an antigen or analyte specific for the presence of COVID-19 antibodies within the patient. The method may use a monoclonal antibody. The antibody may be directed to the spike protein of SARS-CoV-2 or another region of COVID-19. The antibody may have a fluorescent tag. In an embodiment, the body fluid may be exposed to at least one binding antibody, form an antigen-antibody complex. The system and method may determine the presence of the antigen-binding antibody complex. The method and system may be used to determine if a patient has antibodies for the COVID-19 disease. It should be understood that the method and system described herein may be used for diagnostic purposes. In other words, the method may be used as a test kit for example at a medical facility, a testing facility, at home, or the like. The test may be a laminar flow assay. The method may use a fluorescent tag antibody such the level of fluorescent correlates to a level of antibody in the patient body.

At 101, in an embodiment, a method may identify or detect COVID-19. The identification may be rapid in time. The identification may be from a patient's body fluid. The body fluid may be blood, CSF (cerebrospinal fluid), mucus, saliva, or any bodily fluid. The body fluid may be from a patient which may contain COVID-19 antigen, analyte, virions, or the like. An analyte or antigen within the body fluid may be a measure of a antibody protection of a patient. An analyte or antigen may be able to bind to a monoclonal antibody selective for the analyte.

For example, a sample or a body fluid may be withdrawn from a patient using standard medical techniques. Techniques may include sterile cotton swab, blood draw, lumbar puncture, or another accepted form of body fluid collection. Collection may include methodology to preserve the sample such as temperature regulation, sterile techniques, stability agents, buffers, or the like.

At 102, in an embodiment, the body fluid may be exposed to at least one binding antibody. For example, the antibody may be a monoclonal antibody and may selectively bind the spike protein of SARS-CoV-2 or another region of COVID-19. At 103, in an embodiment, the COVID-19 antigen or analyte present in the body fluid may form an antigen-antibody complex with the binding antibody. In an embodiment, detection of viral antigen may be performed using a monoclonal antibody. Alternatively, a method for the detection of immunity may be performed using a secondary antibody which may binds to a primary antibody and antigen in the patient fluid.

In an embodiment, a treatment may be applied to the body fluid. In an embodiment, a treatment may comprise exposing the body fluid to a binding antibody. The binding may be to an antigen specific to the spike protein of SARS-CoV-2. The antigen may include the of SARS-CoV-2 spike glycoprotein. Other antigens may be included for testing as well. Other antigens may include Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex forming antigen-antibody complexes, and combinations thereof. The binding antibody and Covid-19 specific antigen form a antigen-antibody complex.

In an embodiment, the antibodies listed below may be used:

Antibody B16, Mus musculus VH nucleotide sequence:

CAAGTACAGCTGCAGGAGTCTGGACCTGAGCTGGTGAAGCCTGGGGCTT
TAGTGAAGATATCCTGCAAGGCTTCTGGTTACACCTTCACAACCTACGA
TATAAACTGGATGAAGCAGAGGCCTGGACAGGGACTTGAGTGGATTGGA
TGGATTTATCCTGGAGATGGGAGTACAAAGTACAATGAGAAATTCAGGG
GCAAGGTCACACTGACTGCAGACAAATCCTCCAACACAGTCTACATGCA
CCTCATCAGCCTGCCTTCTGAGAAGTCTGCAGTCTATTTCTGTGCAAGA
TCGGTCCTGGGACGGGGGTTTACTTACTGGGGCCAAGGGACTCTGGTCA
CTGTCTCTGCAG,
with an amino acid sequence:
QVQLQESGPELVKPGALVKISCKASGYTFTTYDINWMKQRPGQGLEWIG
WIYPGDGSTKYNEKFRGKVTLTADKSSNTVYMHLISLPSEKSAVYFCAR
SVLGRGFTYWGQGTLVTVSA.

Antibody B16, Mus musculus VL nucleotide sequence:

GACATTGTGATGACACAGACTCCAGCTTCTTTGGCTGTGTCTCTAGGGC
AGAGGGCCACCATATCCTGCAGAGCCAGTGAAAGTGTTGATAGTTATGG
CAATAGTTTTATGCACTGGTACCAGCAGAAACCAGGACAGCCACCCAAA
GTCCTCATCTATTTTGCATCCAACCTAGAATCTGGGGTCCCTGCCAGGT
TCAGTGGCAGTGGGTCTAGGACAGACTTCACCCTCACCATTGATCCTGT
GGAGGCTGATGATGCTGCAACCTATTACTGTCAGCAAAATAATGAGGAT
CCATACACGTTCGGAGGGGGGACCAAGCTGGAAATAAAAC,
with an amino acid sequence:
DIVMTQTPASLAVSLGQRATISCRASESVDSYGNSFMHWYQQKPGQPPK
VLIYFASNLESGVPARFSGSGSRTDFTLTIDPVEADDAATYYCQQNNED
PYTFGGGTKLEIK.

Antibody N12, Mus musculus VH nucleotide sequence:

CAAGTGCAGCTGGAGGAGTCTGGACCTGAGCTGGTGAAGCCTGGGGCTT
TAGTGAAGATATCCTGCAAGGCTTCTGGTTACACCTTCACAAGCTACGA
TATAAACTGGATGAAGCAGAGGCCTGGACAGGGACTTGAGTGGATTGGA
TGGATTTATCCTGGAGATGGTAGTACTAAGTACAATGAGAAATTCAAGG
GCAAGGCCACACTGACTGCAGACAAATCCTCCAGCACAGCCTACATGCA
GATCAGTAGCCTGACTTCTGAAAACTCTGCAGTCTATTTCTGTGCAAGA
TCCGACTTCGGCCACGGGTTTGTTTACTGGGGCCAAGGGACTCTGGTCA
CTGTCTCTGCA,
with an amino acid sequence:
QVQLEESGPELVKPGALVKISCKASGYTFTSYDINWMKQRPGQGLEWIG
WIYPGDGSTKYNEKFKGKATLTADKSSSTAYMQISSLTSENSAVYFCAR
SDFGHGFVYWGQGTLVTVSA.

Antibody N12, Mus musculus VL nucleotide sequence:

GATATTGTGCTCACACAGTCTCCAGCTTCTTTGGCTGTGTCTCTAGGGC
AGAGGGCCACCATATCCTGCAGAGCCAGTGAAAGTGTTGATACTTATGA
CAATAGTTTTATGCACTGGTACCAGCAGAAACCAGGACAGCCACCCAAA
CTCCTCATCTATCTTGCATCCAACCTAGAATCTGGGGTCCCTGCCAGGT
TCAGTGGCAGTGGGTCTAGGACAGACTTCACCCTCACCATTGATCCTGT
GGAGGCTGATGATGCTGCAATCTATTACTGTCAGCAAAATTATGAGGAT
CCGTACACGTTCGGAGGGGGGACCAAGCTGGAAATAAAAC,
with an amino acid sequence:
DIVLTQSPASLAVSLGQRATISCRASESVDTYDNSFMHWYQQKPGQPP
KLLIYLASNLESGVPARFSGSGSRTDFTLTIDPVEADDAAIYYCQQNYE
DPYTFGGGTKLEIK.

In an embodiment, a lateral flow device may be used. A body fluid or a portion of a body fluid may be added to a lateral flow device. In an embodiment, the body fluid may contain an antigen or analyte of COVID-19. The laminar flow device may contain reagents upon a surface. The antigen or analyte may flow via capillary action along a length of the laminar flow device. The laminar flow device may contain the COVID-10 specific antibodies described herein. These antibodies may be referred to as reporter antibodies. The reporter antibodies may migrate to a test line. The laminar flow device may also comprise a test line for a known analyte to confirm proper operation of the laminar flow test. In an embodiment, a Covid-19 antibody may comprise a fluorescent tag. In an embodiment, a fluorescence signal may correlate to the amount of COVID-19 immunity of a patient.

In an embodiment, the spike protein of SARS-CoV-2 antibody may contain an albumin moiety. This antibody may target and rapidly identify COVID-19 antigens. The antibody may or may not include a fluorescent tag. The fluorescent tag may be used for detection techniques. The fluorescent tag may be Alexa-488, Indocyanine green (ICG) or the like.

At 104, in an embodiment, the method may determine the presence or absence of the antigen-antibody complex. The binding antibody may include, for example, a fluorescent antibody, a luminous antibody, combinations thereof, or the like. The concentration of binding antibody may be made as high as necessary for the identification of extremely small, e.g., picogram/microliter, concentrations of the final antigen-antibody complex.

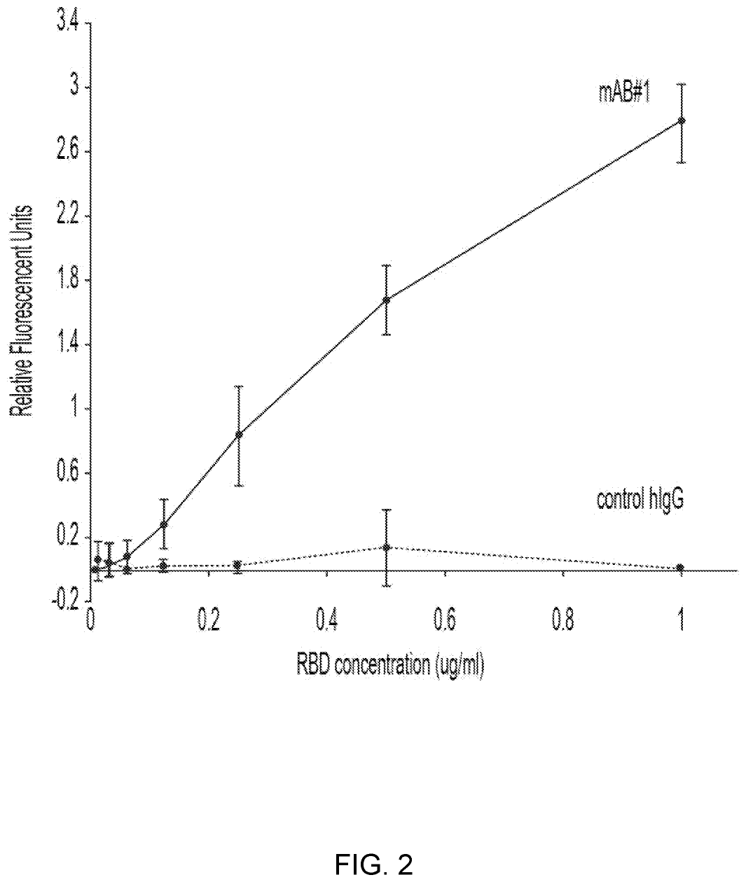

Example binding data are illustrated in FIG. 2. In an embodiment, the antibody may be tagged with a fluorescent indicator. For example, a base human IgG antibody against spike proteins, but not specific to Covid-19 (i.e. does not specifically bind to Covid-19) is illustrated. Also, a fluorescent antibody such as B16 (See sequences described herein) is shown. As an example, the graph shows that the lowest level of RBD concentration that the antibody can detect is 1 nanomolar (6.022×10¹⁴ particles/liter) of the SARS-COV-2 antigen demonstrating a high level of specificity. FIG. 2 illustrates detection of viral antigen (S protein) with antibodies which may be fluorescent antibodies against the viral antigen (S protein). In an embodiment, the method may detect a virus or viral protein in body fluid.

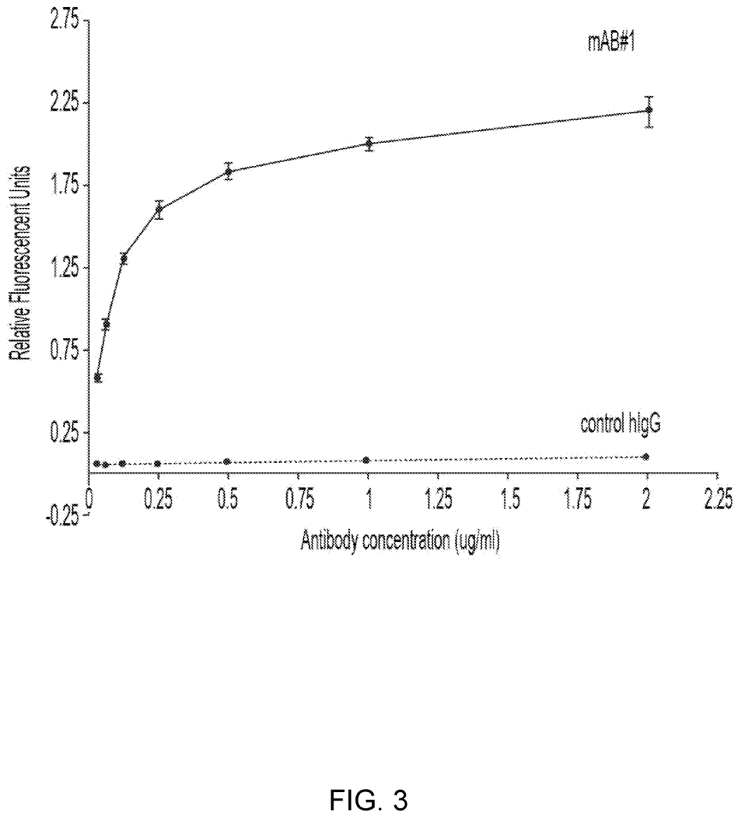

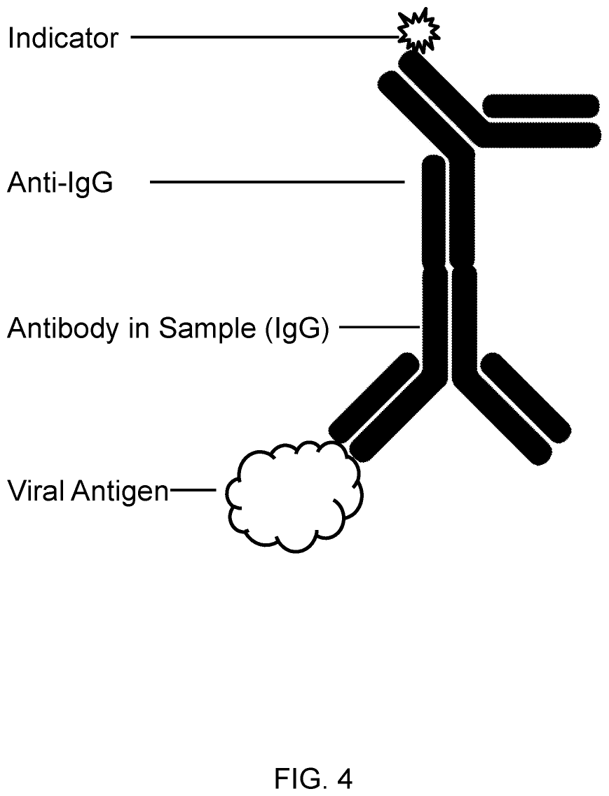

Further example binding data are illustrated in FIG. 3 for a method to detect host antibody immune response to a virus. In other words, to detect antibodies against the S protein. The method may be used to detect immunity of a patient. In an embodiment, the antibody may be labelled with horseradish peroxidase (HRP) or the like. Referring to FIG. 4, in an embodiment, a viral antigen may capture antibodies (primary antibody or antibody in sample IgG) produced in the host or patient body fluid. The S protein-specific antibody (primary antibody) may then be detected with an HRP or fluorescent labelled (indicator) secondary antibody (anti-IgG). As an example, the antibody, and the normal human spike protein antibody (non-specific) is plotted against the actual antibody concentration (as opposed to RBD concentration as in the FIG. 2). The antibody may also be reliably used as an indicator of the presence of the SARS-COV-2 antibody in a sample. This may allow an assessment of whether or not a patient, who has been vaccinated, is developing sufficient antibodies against SARS-COV-2. The antibody may detect the presence of the SARS-COV-2 antibody at levels down to 0.2 nanomolar. This may be used as a test to verify the level of immunity present in an individual. It may show that a patient not only has antibodies, but enough antibodies to determine if the patient needs to be vaccinated or re-vaccinated. The test may be used in a home care or clinical test device to detect the level of antibody protection present in an individual.

In an embodiment, as more antigens are present, the more antibodies will bind and increase the fluorescence signal. Therefore, this antibody may be used as a viral load gradient diagnostic assay. This may allow a physician to determine if a patient has sufficient antibodies, whether they have been vaccinated, if they need a booster, etc. This can be translated into a lateral flow type device. The test may provide a gradient measure of protection, not simply a go-no go test, as exists today. This could screen those who do not need the vaccine due to natural immunity, like those who are asymptomatic. Such a test may direct vaccine resources to a patient with a need for the vaccine.

Additionally or alternatively to lateral flow, the method may utilize different techniques for determining the presence or absence of the antigen-antibody complex. As an example, a dialysis or a variant of dialysis may be used. The dialysis may be used to remove the fluorescent antibody-antigen complex. This may allow for a rapid identification of a COVID-19 sample. Such technique may be automated, controlled by a computer system, or the like. The system may use a threshold, limits, alarms, or the like.

In an embodiment, the method may use flow cytometry analysis of fluorescent labelled antibodies relating to COVID-19. For example, flow cytometry analysis of fluorescent mAbs against SARS-CoV-2 spike (S) protein may be performed. For example, K562 cells may be fixed with 4% PFA (Paraformaldehyde) then permeabilized with 0.1% saponin in PBS (Phosphate-buffered saline). Cells may then be stained with anti-S mAb1 or with mAb1 that has been fluorescently labeled with Alexa488. Stained cells may be processed by flow cytometry. A rightward shift of fluorescent intensity indicates the fluorescent labeling of mAb1.

In an embodiment, a method may utilize a designer fluorescent antibody with an attached macromolecular moiety. The macromolecular moiety, attached to the antibody, may be 1.000 mm to 0.00001 mm in diameter. Disclosed diameters are illustrative and may vary. The antibody-macromolecular moiety-targeted antigen complex would then be blocked for analysis, by using a series of microscreens which contain openings with a diameter 50.00000% to 99.99999% less than the diameter of the designer antibody-macromolecular moiety.

In an embodiment, methodology comprising the removal of the targeted antigen(s)/TA(s) by using a designer fluorescent antibody containing an iron (Fe) moiety. This will then create an Fe-fluorescent Antibody-Antigen (COVID-19/virion) complex. This iron containing complex may then be efficaciously removed using a strong, localized magnetic force field, which may be identified as positive.

In an embodiment, a variant of gel filtration chromatography, which may be utilized for the rapid identification of COVID-19. The fluorescent antibody-target antigen would be used to transport the sample through a size exclusion column that would be used to separate the fluorescent antibody-target antigen by size and molecular weight.

In an embodiment, a methodology using a molecular weight cutoff filtration may be employed. Molecular weight cut-off filtration refers to the molecular weight at which at least or approximately 80% of the target antigen(s)/TA(s) may be prohibited from membrane diffusion.

In an embodiment, a removal methodology for the fluorescent antibody-target antigen(s) may be used. The removal methodology may be selected from a group comprising a mechanical filter, a chemical filter, a dialysis machine, a molecular filter, molecular adsorbent recirculating system (MARS), a plasmapheresis unit, or combinations thereof.

For example, virions may be captured using antibody microarrays. The microarray may contain one or more binding antibody. In an embodiment, the binding antibody may comprise a fluorescent antibody (FI). In another embodiment, the binding antibody may comprise a luminescent (Lu) antibody. The microarray may comprise a plurality of antibodies fixed on a solid surface. The solid surface may be any suitable material. The microarray material may be transparent, such as glass, plastic, silicon, combinations thereof, or the like. The microarray may allow detection of at least one virion antibody complex. The microarray can comprise a plurality of monoclonal antibodies attached at high density on the solid surface. Typically, the microarray may contain millions of antibodies. Exposure of the virion to the binding antibodies on the microarray creates the virion. The complex may be tracked using an appropriate sensor. To identify the virion antibody complex after exposure in the microarrays, the body fluid may then be forced through a container preferably constructed from a transparent material, which exposes the virion antibody complex to a light-sensing device. The sensing device may also create an enlarged, magnified visual image of virion antibody complex. A concentrated and focused intense energy beam, such as light, is then used to properly illuminate the virion antibody complex within the body fluid. Each virion antibody complex may be rapidly identified. The virion antibody complex may also be identified and tracked using optical or digital enhancement or magnification.

At 105, in an embodiment, if an antigen-antibody complex cannot be determined, the system may continue to determine the presence of another antigen-antibody complex in the body fluid. Alternatively, the method or system may determine that the patient body fluid does not contain the antigen or analyte. For example, the patient does not have COVID-19 and/or the patient may not have any immunity to COVID-19. Additionally or alternatively, the system may output an alarm, log an event, or the like. If an antigen-antibody complex can be determined, the system may provide an output at 106. The antigen-antibody complex determination may be an output that is provided to a device in the form of a display, printing, storage, audio, haptic feedback, or the like. Alternatively, or additionally, the output may be sent to another device through wired, wireless, fiber optic, Bluetooth®, near field communication, or the like.

The various embodiments described herein thus represent a technical improvement to the detection of immunity or viral load directed to the spike protein of SARS-CoV-2 or other regions of SARS-CoV-2. Using the techniques as described herein, an embodiment may use a method to determine the presence or absence of SARS-CoV-2 in a sample from a patient.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for detection of viral antigen for the COVID-19 virus, comprising:
obtaining a body fluid from a patient;
introducing the body fluid to at least one binding antibody, wherein the at least one binding antibody binds to an antigen of the SARS-CoV-2 spike (S) protein and comprises an indicator;
forming a viral antigen—antibody complex; and
determining the presence of the viral antigen—antibody complex.

2. The method of claim 1, wherein the at least one binding antibody comprises an isolated monoclonal antibody comprising a VH domain having the amino acid sequence of SEQ. ID No. 2 and a VL domain having the amino acid sequence of SEQ ID. No. 4.

3. The method of claim 1, wherein the at least one binding antibody comprises an isolated monoclonal antibody comprising a VH domain having the amino acid sequence of SEQ. ID No. 6 and a VL domain having the amino acid sequence of SEQ ID. No. 8.

4. The method of claim 1, wherein the body fluid is selected from the group consisting of: blood, cerebrospinal fluid, mucus, and saliva.

5. The method of claim 1, wherein the indicator is selected from the group consisting of: a fluorescent tag and a luminous tag.

6. The method of claim 1, wherein the determining further comprises a viral load gradient diagnostic assay.

7. The method of claim 6, wherein the viral load gradient diagnostic assay determines a treatment plan based upon a quantity of viral antigen—antibody complex in the body fluid.

8. The method of claim 6, wherein the viral load gradient diagnostic assay is correlated to a level of immunity of a patient

9. The method of claim 1, wherein the at least one antibody comprises an iron moiety, wherein a localized magnetic field removes the viral antigen—antibody complex from the body fluid.

10. The method of claim 1, further comprising at least one additional monoclonal antibody from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp?-nsp8 complex, nsp10-nsp14 complex, and nsp10-nsp16 complex forming antigen-antibody complexes.

11. A method for detection of immunity for the COVID-19 virus, comprising:
obtaining a body fluid from a patient, wherein the body fluid comprises a viral antigen—primary antibody complex of SARS-CoV-2 spike protein;
introducing the body fluid to at least one secondary antibody, wherein the at least one secondary antibody binds to the primary antibody of the SARS-CoV-2 spike (S) protein and an indicator;
forming an antigen—primary antibody—secondary complex; and
determining the presence of the antigen—primary antibody—secondary complex, wherein the indicator correlates to a level of immunity of the patient.

12. The method of claim 11, wherein the primary antibody comprises an isolated monoclonal antibody comprising a VH domain having the amino acid sequence of SEQ. ID No. 2 and a VL domain having the amino acid sequence of SEQ ID. No. 4.

13. The method of claim 11, wherein the primary antibody comprises an isolated monoclonal antibody comprising a VH domain having the amino acid sequence of SEQ. ID No. 6 and a VL domain having the amino acid sequence of SEQ ID. No. 8.

14. The method of claim 11, wherein the body fluid is selected from the group consisting of: blood, cerebrospinal fluid, mucus, and saliva.

15. The method of claim 11, wherein the indicator is selected from the group consisting of: a fluorescent tag and a luminous tag.

16. The method of claim 11, wherein the determining further comprises a viral load gradient diagnostic assay.

17. The method of claim 16, wherein the viral load gradient diagnostic assay determines a treatment plan based upon a quantity of viral antigen—antibody complex in the body fluid.

18. The method of claim 16, wherein the viral load gradient diagnostic assay is correlated to a level of immunity of a patient

19. The method of claim 11, wherein the primary antibody comprises an iron moiety, wherein a localized magnetic field removes the viral antigen—antibody complex from the body fluid.

20. A method for detection of immunity for the COVID-19 virus, comprising:
obtaining a body fluid from a patient;
introducing the body fluid to a lateral flow device comprising at least one binding antibody, wherein the at least one binding antibody binds to an antigen of the SARS-CoV-2 spike (S) protein and comprises a fluorescent tag;
forming an antigen-antibody complex; and
determining the presence of the antigen-antibody complex, wherein a fluorescent signal correlates to a level of immunity of the patient.


SEQUENCE LISTING (8 sequences)

SEQ ID NO: 1 - DNA, 355 bp, Mus musculus
caagtacagctgcaggagtctggacctgagctggtgaagcctggggctttagtgaagatatcctgcaaggcttctggttacaccttcacaacctacgatataaactggatgaagcagaggcctggacagggacttgagtggattggatggatttatcctggagatgggagtacaaagtacaatgagaaattcaggggcaaggtcacactgactgcagacaaatcctccaacacagtctacatgcacctcatcagcctgccttctgagaagtctgcagtctatttctgtgcaagatcggtcctgggacgggggtttacttactggggccaagggactctggtcactgtctctgcag

SEQ ID NO: 2 - protein, 118 aa, Mus musculus
QVQLQESGPELVKPGALVKISCKASGYTFTTYDINWMKQRPGQGLEWIGWIYPGDGSTKYNEKFRGKVTLTADKSSNTVYMHLISLPSEKSAVYFCARSVLGRGFTYWGQGTLVTVSA

SEQ ID NO: 3 - DNA, 334 bp, Mus musculus
gacattgtgatgacacagactccagcttctttggctgtgtctctagggcagagggccaccatatcctgcagagccagtgaaagtgttgatagttatggcaatagttttatgcactggtaccagcagaaaccaggacagccacccaaagtcctcatctattttgcatccaacctagaatctggggtccctgccaggttcagtggcagtgggtctaggacagacttcaccctcaccattgatcctgtggaggctgatgatgctgcaacctattactgtcagcaaaataatgaggatccatacacgttcggaggggggaccaagctggaaataaaac

SEQ ID NO: 4 - protein, 111 aa, Mus musculus
DIVMTQTPASLAVSLGQRATISCRASESVDSYGNSFMHWYQQKPGQPPKVLIYFASNLESGVPARFSGSGSRTDFTLTIDPVEADDAATYYCQQNNEDPYTFGGGTKLEIK

SEQ ID NO: 5 - DNA, 354 bp, Mus musculus
caagtgcagctggaggagtctggacctgagctggtgaagcctggggctttagtgaagatatcctgcaaggcttctggttacaccttcacaagctacgatataaactggatgaagcagaggcctggacagggacttgagtggattggatggatttatcctggagatggtagtactaagtacaatgagaaattcaagggcaaggccacactgactgcagacaaatcctccagcacagcctacatgcagatcagtagcctgacttctgaaaactctgcagtctatttctgtgcaagatccgacttcggccacgggtttgtttactggggccaagggactctggtcactgtctctgca

SEQ ID NO: 6 - protein, 118 aa, Mus musculus
QVQLEESGPELVKPGALVKISCKASGYTFTSYDINWMKQRPGQGLEWIGWIYPGDGSTKYNEKFKGKATLTADKSSSTAYMQISSLTSENSAVYFCARSDFGHGFVYWGQGTLVTVSA

SEQ ID NO: 7 - DNA, 334 bp, Mus musculus
gatattgtgctcacacagtctccagcttctttggctgtgtctctagggcagagggccaccatatcctgcagagccagtgaaagtgttgatacttatgacaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaatctattactgtcagcaaaattatgaggatccgtacacgttcggaggggggaccaagctggaaataaaac

SEQ ID NO: 8 - protein, 111 aa, Mus musculus
DIVLTQSPASLAVSLGQRATISCRASESVDTYDNSFMHWYQQKPGQPPKLLIYLASNLESGVPARFSGSGSRTDFTLTIDPVEADDAAIYYCQQNYEDPYTFGGGTKLEIK